(19) United States
(12) Patent Application Publication
(10) Pub. No.: US 2004/0087963 A1
(43) Pub. Date: May 6, 2004

(54) Medical device having increased lubricity

(76) Inventors: Alexei Ossipov, Aliso Viejo, CA (US); Xiugao Liao, Irvine, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/214,219

(22) Filed: Aug. 7, 2002

(51) Int. Cl. A61F 9/00

(52) U.S. Cl. 606/107

(57) ABSTRACT

A cartridge having increased lubricity and a method used to increase the lubricity of medical devices such as a lens cartridge. The inner surface of a lens cartridge formed from polypropylene and at least one other constituent is exposed to a treatment gas, which may be a mixture of steam and carbon dioxide gas, humidified carbon dioxide, sulfur dioxide, nitrogen dioxide or a combination thereof, to increase the lubricity of an inner surface of the lens cartridge or medical device. Various processing conditions may be controlled to control the amount of increased lubricity.

### FIELD OF THE INVENTION

The present invention is directed to improving the surface lubricity of medical devices. More particularly, the invention relates to increasing the surface lubricity of a polypropylene lens cartridge for use with surgical instruments for implantation of a deformable intraocular lens into an eye.

### BACKGROUND OF THE INVENTION

The physiology of the human eye includes an anterior chamber located between the cornea, or outer surface of the clear part of the eye, and the iris, the pigmented portion of the eye that is responsive to light, and a posterior chamber, filled with vitreous humor. A crystalline lens, which includes a lens matrix contained within a capsular bag, is located behind the iris and separates the iris from the posterior chamber. The crystalline lens is attached to the ciliary muscle by cord-like structures called zonules. Lining the rear of the posterior chamber is the retina, the light sensing organ of the eye, that is an extension of the optic nerve.

As the natural crystalline lens ages, the structure of the lens matrix of the crystalline lens changes, becoming hazy and relatively inflexible. Eventually, the hazing of the lens matrix may progress to the point where the lens is considered cataractous, which may seriously occlude the amount of light passing through the crystalline lens and ultimately onto the retina. Fortunately, modem surgical techniques have been developed which allow removal of the cataractous lens and implantation of an artificial lens or intraocular lens.

Deformable intraocular lenses made from silicone, soft acrylics and hydrogels have become widely used because of the ability to fold these lenses and insert them through a relatively small incision in the eye. One method of inserting a deformable intraocular lens into an eye is by using a lens cartridge with a surgical lens injecting device. The intraocular lens folds inside the lens cartridge and is then pushed through a relatively small diameter lumen through which the lens is then implanted into the eye. Examples of such lens cartridge and injecting devices can be found in U.S. Pat. Nos. 5,494,484, 5499,987 and 5,772,666, the entire contents of which are incorporated herein by reference.

One difficulty with presently available systems is that a deformable lens must be ejected from a cartridge through a relatively small incision in the sclera or cornea of the eye. Accordingly, a relatively large intraocular lens must be folded or rolled so that it fits within a passageway in the cartridge of a sufficiently small diameter so that the nozzle portion of the cartridge will fit within the small incision the physician made in the eye. In present systems, there may be sufficient frictional force between the inner surface of the passageway and the intraocular lens such that the lens may be damaged when it is forced through the passageway and ejected out of the cartridge and into the eye. One approach to reducing the frictional between the inner surface of the passageway and the lens has been to package the cartridge in a manner which maintains a level of hydration of the inner surface of the passageway.

One problem with this approach, however, is that current lens cartridges tend to dry-out due to water evaporation over time in storage, causing these lens cartridges to no longer have a high surface lubricity. When the surface lubricity of the lens cartridges decreases, so does their performance in use. As stated above, when the lens cartridge drys out, the intraocular lens can be damaged as it is being pushed out of the cartridge and into the eye. Tearing of the intraocular lens can even occur as it is ejected from an over dried lens cartridge that does not have the required surface lubricity to better allow the intraocular lens to slide out through the passageway of the cartridge. These problems can also occur with the use of a new lens cartridge that does not initially have a high surface lubricity.

What has been needed and heretofore unavailable, is a method of increasing or restoring the surface lubricity of lens cartridges. Lens cartridges need to have a high surface lubricity in order to protect the intraocular lens that is sliding through the lens cartridge as it is being pushed out into the eye by the injecting device. The devices and methods of the present invention satisfy this need.

### SUMMARY OF THE INVENTION

The present invention provides a medical device, such as a lens cartridge for use in implanting a deformable intraocular lens, that has an increased surface lubricity.

In one aspect, the present invention is embodied in a lens cartridge formed from polypropylene and at least one other constituent. After the lens cartridge is sterilized, the inner surface of the lens cartridge may be activated by exposing the inner surface to a mixture of steam and carbon dioxide. Alternatively, the inner surface may be activated by exposing the inner surface to humidified carbon dioxide, that is, for example, carbon dioxide that has been bubbled through water to increase the partial pressure of water vapor within the gas stream.

In an other embodiment of the system and methods of the present invention, a lens cartridge for use in a surgical lens inserting device for implantation of a deformable intraocular lens may be treated to increase the surface lubricity of a passageway within the cartridge. In this embodiment, the lens cartridge may be placed in a chamber. A mixture of steam and carbon dioxide are injected into the chamber for a selected period of time. The steam and carbon dioxide interact with the surfaces of the lens cartridge to increase the lubricity of the surfaces. Alternatively, humidified carbon dioxide may be used to treat the surfaces of the lens cartridge. In yet another embodiment, the treatment gas may be selected from the group consisting of carbon dioxide, sulfur dioxide and nitrogen dioxide. The treatment gas may be either mixed with steam or it may be humidified using methods well known to those skilled in the relevant art. In still another embodiment, the treatment gas may be a combination of two or more gases selected from the group consisting of carbon dioxide, sulfur dioxide and nitrogen dioxide. As before, this combination may be mixed with steam or humidified using well known methods to increase the partial pressure of water vapor in the gas stream.

In a further embodiment, the present invention includes a lens cartridge made of polypropylene that has been modified with glycerin monostearate and sterilized using ethylene oxide. The lens cartridge is placed into a chamber where a treatment gas including a mixture of carbon dioxide gas and steam is injected into the chamber for a certain period of time. Alternatively, the treatment gas may be humidified carbon dioxide, sulfur dioxide, nitrogen dioxide, or some combination thereof. In one embodiment, the chamber has an initial pressure within the chamber after the lens cartridge is placed within the chamber. The pressure inside the chamber may be reduced to a reduced pressure that is less than the initial pressure. The treatment gas can be injected into the chamber in such a manner that the reduced pressure is maintained during the selected treatment time. Alternatively, the pressure within the chamber may be controlled during injection of the treatment gas such that the pressure within the chamber increases to selected increased pressure that is greater than the reduced pressure; the pressure may also be increased such that the pressure within the chamber during the treatment process is greater than the initial pressure within the chamber.

In yet another embodiment, the temperature within the chamber during treatment may be controlled to a selected temperature. For example, in one embodiment, the temperature of the chamber is maintained at 30° C.

In another embodiment where the treatment gas is a mixture of, for example, steam and carbon dioxide, the mixture may be injected for a first selected period of time. The steam injection is then halted, while carbon dioxide continues to be injected for a further selected period of time.

In a further embodiment, the lens cartridge may be placed in an unsealed hermitically sealable package, such as a foil pouch, before the lens cartridge is placed in the chamber. The lens cartridge and pouch are then treated in accordance with the methods of the present invention described above. At the completion of the treatment process, the lens cartridge and unsealed hermetically sealable package is removed from the chamber and the hermetically sealable package is sealed. Sealing the package assists in preventing degradation of the increased lubricity of the surfaces of the lens cartridges when the sealed packages are stored for a prolonged period of time before use.

In a still further embodiment of the present invention, the treatment process may be carried out more than once on a lens cartridge to optimize the increased lubricity of the surfaces of the cartridge. For example, a lens cartridge may be treated two or more times to provide an optimal level of lubricity.

Other features and advantages of the present invention will become more apparent from the following detailed description, taken in conjunction with the accompanying drawings.

### DESCRIPTION OF THE DRAWINGS

FIG. 1 is a perspective view of a lens cartridge shown in the open position to allow a deformable intraocular lens to be loaded therein;

FIG. 2 is a FT-IR spectrum comparing a lens cartridge before and after treatment using the present method; and

FIGS. 3A and 3B are diagrams showing the contact angle of a liquid droplet with the surface after treatment in accordance with the principles of the present invention and prior to treatment, respectively.

### DETAILED DESCRIPTION OF THE PREFERRED EMBODIMENTS

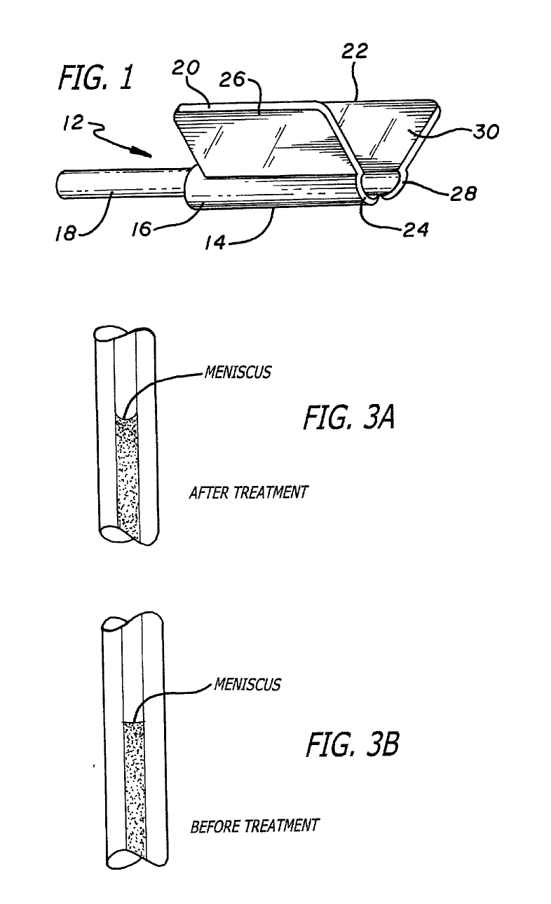

A lens cartridge or microcartridge is used in connection with a lens injecting device for inserting a deformable intraocular lens into an eye of a patient through a relatively small incision made in the ocular tissue. One embodiment of a lens cartridge 12 is shown in FIG. 1. The lens cartridge 12 comprises a split tubular member 14 extending to a continuous tubular member or lens holding portion 16 and an implantation nozzle portion 18. In a closed position, the lens cartridge has a continuous circular or oval passageway of the same diameter extending through the split tubular member 14, the continuous tubular member 16, and the implantation nozzle portion 18. The split tubular member 14 is defined by a fixed portion 20 and a movable portion 22. The fixed portion 20 is fixed relative to the implantation nozzle portion 18, and is defined by a tubular portion 24 and extension 26. The movable portion 22 is movable relative to the fixed portion 20 for opening and closing the split tubular member 14, and the movable portion 22 is defined by a tubular portion 28 and extension 30.

Further details and alternative embodiments of a lens cartridge can be found in U.S. Pat. No. 5,499,987 issued to Feingold.

The lens cartridge 12 is preferably made of an injection molded plastic such as polypropylene. Lens cartridges made of polypropylene should contain a releasing agent such as glycerin monostearate (“GMS”) and an anti-static agent such as stearamide on the surface. Another anti-static agent known as monostearylamide may also be found on the surface.

It is known that polypropylene has a glass transition temperature as low as—13° C. Above—13° C., GMS molecules can migrate from a center layer of the lens cartridge to a near surface layer and vice-versa. The migration direction of the GMS molecules depends on storage conditions of the polypropylene lens cartridge. Conditions including relatively high temperature and humidity will induce the GMS molecules to migrate to the surface region and near surface region of the polypropylene lens cartridges. However, relatively low temperature and dry conditions will induce the GMS molecules to migrate to the center layer of the polypropylene lens cartridges. Keeping the GMS molecules at surface or near surface regions of the polypropylene lens cartridge will help maintain its surface lubricity. It is very important to maintain the surface lubricity of the lens cartridge so that in use a lens will easily slide through the cartridge and into the eye of the patient, with a less likely chance of complications.

During manufacturing of lens cartridges, in order to maintain a good amount of the releasing agent GMS on the surface of the cartridge, high pressure molding parameters should be used, which is usually associated with a low mold temperature of about 5°-10° C. In one embodiment, high pressure injection may be used to mold the cartridges at a very low molding temperature. The molding temperature must be below 205° C. because GMS will start to decompose (oxidize) above this temperature which will provide a very low lubricity on the surface of the cartridge. With this embodiment, the lens cartridge should be softer than a normally prepared cartridge, which indicates a more amorphous fraction (the cartridge will expand without breaking) in the casting polymer when compared to the normally prepared cartridge. However, the friction parameter of the lens cartridge will be very low respectively, and will allow the cartridge to perform well during operation. During the injection molding process, additional GMS may also be added, up to 0.7% by weight, to assist in maintaining the level of GMS on the surface of the cartridge at a level sufficient to ensure that the coefficient of friction of the cartridge surface region does not increase to unacceptable levels.

The present invention includes a method of treating the surface of a sterilized GMS modified polypropylene lens cartridge with carbon dioxide in the presence of water to improve the lubricity of the surface. The carbon dioxide gas may react with the glycerol end of the GMS molecules and generates a carbonic acid group at the surface of the polypropylene lens cartridge. Carbon dioxide also influences migration of the GMS molecules to the surface or near surface regions of the polypropylene lens cartridge. As illustrated by the results of contact angle testing shown in FIG. 3, this method of treating the surface of the cartridge provides for a much higher surface hydrophilicity of the polypropylene lens cartridge. As shown in FIG. 3B, cartridge material before treatment has a coefficient of friction of approximately 0.200. When the untreated cartridge comes into contact with water, a meniscus forms at the solid/liquid interface. The curvature of the meniscus is an indication of the hydrophilicity, or affinity for water or moisture of the surface. In FIG. 3B, the meniscus has very little curvature, indicating that the surface of the untreated cartridge is relatively hydrophobic. In contrast, the meniscus shown in FIG. 3A formed at the interface between the surface of a treated cartridge and the liquid is more highly curved, indicating that the treated cartridge surface is more hydrophilic that the surface of the untreated cartridge. Additionally, the coefficient of friction of treated cartridge surface is 0.035, substantially less, and thus more slippery, than the untreated surface. It will thus be understood by one skilled in the relevant art that the surface lubricity of the lens cartridge is significantly improved compared to a lens cartridge before the treatment.

The method of one embodiment of the present invention increases the surface lubricity of GMS modified polypropylene lens cartridges and helps restore high surface lubricity to over dried lens cartridges. In this embodiment, the polypropylene lens cartridge is placed into a chamber and carbon dioxide gas and steam are injected into the chamber for a certain amount of time. In an alternative embodiment, humidified carbon dioxide gas, that is, carbon dioxide that has been bubbled through water to increase the partial pressure of water vapor in the gas mixture, is injected into the chamber and the cartridges are treated at a selected temperature for a selected period of time.

The pressure within the chamber may be varied, depending on the parameters of the process that are selected. For example, the chamber may be maintained at atmospheric pressure, or the pressure in the chamber may be lowered by means of a vacuum pump or other means well known in the art. Alternatively, the pressure within the chamber may be controlled so that the pressure during treatment of the cartridges may be greater than atmospheric pressure. In any case, the steam and carbon dioxide gas, or humidified carbon dioxide gas, are injected into the chamber and suitable controls, such as pressure valves, flow controls and the like are used to control the internal pressure of the chamber during injection to maintain the pressure within the chamber at the desired level.

A single treatment has been found to increase the surface lubricity of a cartridge. The inventors have further determined that treating the cartridges three times, that is carrying out the methods of the present invention described above on a cartridge three times, provides a substantial increase in the surface lubricity of the cartridge resulting in easier insertion of an intraocular lens through the cartridge during use. However, in cases where a lesser increase in surface lubricity of the cartridge is needed, the process may be repeated fewer times. It will also be understood that the process maybe carried out on a cartridge more than three times without damaging the cartridge or rendering the cartridge otherwise unusable; accordingly multiple processing of cartridges using the methods of the present invention described above are within the scope contemplated by the present invention.

While the methods of various embodiments of the present invention have bee described above with reference to the injection of carbon dioxide gas, it will be understood that other gases may also be used. For example, sulfur dioxide and nitrogen dioxide may be used. The sulfur dioxide or nitrogen dioxide may also be injected into the chamber along with steam, or they may be humidified by bubbling the gases through water or by other methods well known in the art. Moreover, it is contemplated that the carbon dioxide, sulfur dioxide and nitrogen dioxide may be used alone, or in combination, during the treatment process.

In typical use, cartridges are sterilized before they are placed in the chamber and treated using the methods of the present invention. Generally, cartridges formed from polypropylene are sterilized with ethylene oxide (“ETO”). The inventors have observed that when sterilized cartridges are placed in the chamber and subjected to carbon dioxide gas and steam, or humidified carbon dioxide gas, the GMS present on the surface of the cartridge reacts with the ETO and forms poly(ethyleneglycoloxy) glycerol monostearate. “(PGM”). One useful property of PGM is that it is more slippery than GMS.

When the lens cartridge is exposed to the mixture of carbon dioxide and water, an acidic reaction occurs which promotes the reaction between GMS and ETO. In the presence of ETO, GMS absorbs water and creates a micelle, which will likely have a flat or spherical shape. The hydrophobic ends of the GMS molecules will be surrounded by other hydrophobic ends from other GMS molecules while the hydrophilic ends will be surrounded by water.

Utilizing the methods of the present invention, molecules of carbon dioxide dissolve into water molecules present on the surface of a polypropylene cartridge to form H₂CO₃ on the surface of the cartridge. Moreover, the carbon dioxide molecules interpenetrate into the near surface layer or center layer of the polypropylene cartridges during the treatment process and interact with adsorbed water within the matrix of the polypropylene cartridge to form H₂CO₃. It is known in the art that H₂CO₃ can form a hydrogen bond with a water molecule already linked to the hydrophilic end of a GMS molecule. This hydrogen bond will significantly increase the hydrophilicity of the GMS molecule by increasing the driving force of the GMS molecules to migrate from the center layer to the near surface layer to the surface of the cartridge. Carbon dioxide molecules may also react with GMS molecules to form trace amounts of GMS-carbonic acid, which acts as a surfactant to increase the surface lubricity of the polypropylene cartridge. After the lens cartridges have been treated with this method, they are repackaged into a foil pouch designed to prevent evaporation of water from the inside of the package during storage. Storing the treated cartridges in this manner prolongs the shelf life of increased surface lubricity of the treated cartridges.

The following are nine exemplary treatment procedures embodying various aspects of the present invention that may be used to treat cartridges to increase their surface lubricity and hydrophilicity. It will be understood that these are examples only, and that the scope of the present invention is not limited to these embodiments.

Treatment Procedure 1

About 50 (although the amount of cartridges can vary widely) sterilized polypropylene lens cartridges are placed into individual foil pouches, which are open on at least one end or side. The cartridges and foil pouches are then placed onto a rack with the open end or side of the pouch facing up. The rack is then placed into a chamber with the open side or end of the foil pouch facing towards the top of the chamber. When the rack is inside the chamber, a vacuum pump is turned on and the pressure inside the chamber is decreased in a controlled manner until the pressure within the chamber reaches about 360±50 torr. A mixture of steam and carbon dioxide gas are then injected into the chamber for at least two minutes. The injection of carbon dioxide gas and steam is controlled, in conjunction with the vacuum pump, to allow injection of the gas mixture while maintaining the pressure within chamber at 360±50 torr. During the treatment time, the temperature within the chamber is monitored and controlled so that the temperature within the chamber is approximately 30° C. After the desired treatment time has elapsed, the vacuum pump is turned off. The injection of carbon dioxide gas into the chamber is continued for at least four minutes causing the pressure to increase within the chamber. Typically, the continued injection of carbon dioxide gas results in the pressure increasing until the chamber is at a positive pressure, that is, a pressure exceeding atmospheric pressure. The gas injection system is then turned off, and, after waiting a minimum of four minutes, the pressure is released from the chamber. This process may be repeated two more times, although improved lubricity of the polypropylene lens cartridge is observed after a single treatment. When the desired number of treatments have been accomplished, the rack hold the cartridges is removed from the chamber, and the foil pouch is scaled immediately to prevent water evaporation from the pouch.

Treatment Procedure 2

About 50 (although the amount of cartridges can vary widely) sterilized polypropylene lens cartridges are placed into individual foil pouches, which are open on at least one end or side. The cartridges and foil pouches are then placed onto a rack with the open end or side of the pouch facing up. The rack is then placed into a chamber with the open side or end of the foil pouch facing towards the top of the chamber. When the rack is inside the chamber, a mixture of steam and carbon dioxide gas is injected into the chamber for at least twenty minutes. The pressure with the chamber, and the flow of gas into the chamber are controlled such that the chamber is maintained at approximately atmospheric pressure during this phase of the treatment.

After the selected period time during which steam and carbon dioxide are being injected has elapsed, the injection of steam is halted, and carbon dioxide gas is continued to be injected into the chamber. During this phase of the treatment, the pressure within the chamber is controlled by adjusting the flow of gas into and out of the chamber such that the pressure within the chamber increases to approximately 775.7 torr. The pressure is maintained at approximately 775.7 torr for at least one hour and the temperature within the chamber is controlled to maintain the temperature within the chamber at approximately 30° C. After this time has elapsed, injection of carbon dioxide gas is halted and the chamber is vented, decreasing the pressure within the chamber until the pressure within the chamber reaches equilibrium with the atmospheric pressure outside of the chamber. The process may be repeated two more times, although improved lubricity of the polypropylene lens cartridge is observed after a single treatment. When the desired number of treatment cycles have been completed, the rack holding the cartridges and foil pouches is removed from the chamber, and the foil pouch is sealed immediately to prevent water evaporation from the pouch.

Treatment Procedure 3

About 50 (although the amount of cartridges can vary widely) sterilized polypropylene lens cartridges are placed into individual foil pouches, which are open on at least one end or side. The cartridges and foil pouches are then placed onto a rack with the open end or side of the pouch facing up. The rack is then placed into a chamber with the open side or end of the foil pouch facing towards the top of the chamber. When the rack is inside the vacuum steam and carbon dioxide gas are injected into the chamber for at least forty-five minutes. After this period of time, the injection of steam is halted, while carbon dioxide gas continues to pass through the chamber for at least another two hours. During this treatment time, the pressure of carbon dioxide gas within the chamber is controlled so that the pressure within the chamber remains at approximately atmospheric pressure and the temperature within the chamber is controlled to maintain the temperature within the chamber at approximately 30° C. The process may then be repeated at least two more times, although improved lubricity of the polypropylene lens cartridge is observed after a single treatment. When the desired number of treatment cycles have been completed, the rack holding the cartridges and foil pouches is removed from the chamber, and the foil pouch is sealed immediately to prevent water evaporation from the pouch.

Treatment Procedure 4

About 50 (although the amount of cartridges can vary widely) sterilized polypropylene lens cartridges are placed into individual foil pouches, which are open on at least one end or side. The cartridges and foil pouches are then placed onto a rack with the open end or side of the pouch facing up. The rack is then placed into a chamber with the open side or end of the foil pouch facing towards the top of the chamber. When the rack is inside the chamber, the chamber is sealed and a vacuum pump is used to reduce the pressure within the chamber to approximately 360±40 torr. The temperature within the chamber is monitored and controlled so as to maintain the temperature within the chamber at about 30° C. Once the pressure is reduced to 360±40 torr, this reduced pressure is maintained for about two minutes. The vacuum pump is turned off, and humidified carbon dioxide gas is injected into the chamber for at least four minutes.

After the selected period time during which humidified carbon dioxide gas is being injected has elapsed, the injection is halted. If desired, the process may then be repeated by turning on the vacuum pump and reducing the pressure within the chamber to 360±40 torr, maintaining that reduced pressure for about two minutes, turning offthe vacuum and repeating the injection process. Typically, the treatment process is repeated at least four times, although improved lubricity of the polypropylene lens cartridge is observed after a single treatment. When the desired number of treatment cycles have been completed, the rack holding the cartridges and foil pouches is removed from the chamber, and the foil pouch is sealed immediately to prevent water evaporation from the pouch.

Treatment Procedure 5

About 50 (although the amount of cartridges can vary widely) sterilized polypropylene lens cartridges are placed into individual foil pouches, which are open on at least one end or side. The cartridges and foil pouches are then placed onto a rack with the open end or side of the pouch facing up. The rack is then placed into a chamber with the open side or end of the foil pouch facing towards the top of the chamber. When the rack is inside the chamber, the chamber is sealed and a vacuum pump is used to reduce the pressure within the chamber to approximately 360±40 torr. The temperature within the chamber is monitored and controlled so as to maintain the temperature within the chamber at about 30° C. Once the pressure is reduced to 360±40 torr, steam and carbon dioxide gas is injected into the chamber for at least two minutes. During the injection process, the pressure within the chamber is controlled so as to maintain a pressure of 360±40 torr.

After the selected period time during which the steam and carbon dioxide gas are being injected into the chamber has elapsed, the vacuum pump and steam injection is stopped. Injection of carbon dioxide gas is continued for at least a further four minutes. If desired, the process may then be repeated by turning on the vacuum pump and reducing the pressure within the chamber to 360±40 torr, and repeating the injection process described above. Typically, the treatment process is repeated at least two times, although improved lubricity of the polypropylene lens cartridge is observed after a single treatment. When the desired number of treatment cycles have been completed, the rack holding the cartridges and foil pouches is removed from the chamber, and the foil pouch is sealed immediately to prevent water evaporation from the pouch.

Treatment Procedure 6

About 50 (although the amount of cartridges can vary widely) sterilized polypropylene lens cartridges are placed onto a rack. The rack is then placed into a chamber. When the rack is inside the vacuum steam and carbon dioxide gas are injected into the chamber for at least twenty minutes. After this period of time, the injection of steam is halted, while carbon dioxide gas continues to be injected until the chamber pressure is approximately 775.7 torr. The flow of carbon dioxide gas into the chamber is controlled to maintain the chamber pressure at about 775.7 torr for at least 1 hour. The temperature within the chamber is controlled to maintain the temperature within the chamber at approximately 30° C. during the treatment process. At the end of the one hour duration, carbon dioxide injection is turned off and the pressure within the chamber is released, allowing the pressure in the chamber to equalize with the atmospheric pressure outside of the chamber. The process may then be repeated at least two more times, although improved lubricity of the polypropylene lens cartridge is observed after a single treatment. When the desired number of treatment cycles have been completed, the rack holding the cartridges is removed from the chamber and the cartridges are placed into foil pouches. The foil pouches are sealed immediately to prevent water evaporation from the pouches.

Treatment Procedure 7

About 50 (although the amount of cartridges can vary widely) sterilized polypropylene lens cartridges are placed onto a rack. The rack is then placed into a chamber. When the rack is inside the chamber, the chamber is closed and steam and carbon dioxide gas are injected into the chamber for at least forty-five minutes. The steam injection is halted and carbon dioxide gas is allowed to pass through the chamber for at least another 2 hours. Typically, the treatment process is repeated at least two more times, although improved lubricity of the polypropylene lens cartridge is observed after a single treatment. When the desired number of treatment cycles have been completed, the rack holding the cartridges is removed from the chamber and the cartridges are placed into foil pouches. The foil pouches are sealed immediately to prevent water evaporation from the pouches.

Treatment Procedure 8

About 50 (although the amount of cartridges can vary widely) sterilized polypropylene lens cartridges are placed into individual foil pouches, which are open on at least one end or side. The cartridges and foil pouches are then placed onto a rack with the open end or side of the pouch facing up. The rack is then placed into a chamber with the open side or end of the foil pouch facing towards the top of the chamber. When the rack is inside the chamber, the chamber is closed and humidified carbon dioxide gas is injected into the chamber for approximately one and a half hours.

After the selected period time during which the humidified carbon dioxide gas is being injected into the chamber has elapsed, the chamber is opened and the rack holding the cartridges in their foil pouches is removed from the chamber. The foil pouches are sealed immediately after removal from the chamber to prevent water evaporation from the pouch.

Treatment Procedure9

About 50 (although the amount of cartridges can vary widely) sterilized polypropylene lens cartridges are placed onto a rack. The rack is then placed into a chamber. When the rack is inside the chamber, the chamber is closed and humidified carbon dioxide gas is injected into the chamber for approximately one and a half hours.

After the selected period time during which the humidified carbon dioxide gas is being injected into the chamber has elapsed, the chamber is opened and the rack holding the cartridges is removed from the chamber. The treated cartridges are placed into individual foil pouches which are sealed immediately to prevent water evaporation from the pouches.

Following the treatment of the present invention, the polypropylene lens cartridges showed good lubricity. Ejection and anti-scratch tests were conducted before and after the treatment to confirm the results. The results of the ejection test are listed in Table 1, and the anti-scratch slippery test results are listed in Table 2 below.

TABLE 1
EJECTION TEST RESULTS

| Parameter | | | | |
|---|---|---|---|---|
| Cartridge | Before Treatment | | After Treatment | |
| Model | Lens Diopter | E-Result | Lens Diopter | E-Result |
| AQ2.8s | 10.0-30.0 D | 4/80* | 10.0-30.0 D | 0/80 |
| ST-45s | 10.0-30.0 D | 6/80* | 10.0-30.0 D | 0/80 |
| MTC-60c | 10.0-30.0 D | 4/80* | 10.0-30.0 D | 0/80 |

*Number of lenses torn out of 80 lenses tested.

This data in Table 1 shows that untreated cartridges have a higher probability of damaging an intraocular lens during ejection of the lens from the cartridge than lens cartridges after treatment. Before treatment, the cartridges may be dried-out, in which case they have a lower surface lubricity, or they may be new cartridges that did not have sufficient surface lubricity initially. The E-result column of Table 1 shows that at least 4 out of the 80 lenses tested (no matter what model of cartridge was used) were torn when ejected out of a lens cartridge before treatment, while none of the lenses ejected from treated lens cartridges were torn. These tests confirm that treatment in accordance with the methods of the present invention provides lens cartridges with increased surface lubricity, and that intraocular lenses ejected from cartridges with higher surface lubricity are less likely to be damaged than lenses ejected from untreated cartridges. This improved ability to eject lenses without damaging them is advantageous in that lenses damaged during ejection into the eye typically must be removed from the eye, as they will most likely not function properly or may in fact damage sensitive ocular tissues. Thus, cartridges treated in accordance with the methods of the present invention will aid in reducing operation time, by virtually eliminating the need to remove a damaged lens, provide increased confidence to the implanting physician, and reduce the amount of force required to eject a lens from the cartridge.

Table 2 contains the results of a test designed to measure the slipperiness of surfaces treated in accordance with the methods of the present invention compared to untreated surfaces. In this test, an indenter is drawn across the surface. As the indenter is drawn across the surface, the friction force, or stickiness, of the surface is measured and the coefficient of friction for the surface is determined.

TABLE 2
ANTI-SCRATCH SLIPPERY TEST RESULTS

| | | |
|---|---|---|
| | Maximum | Average On 1^{st} |
| Test Condition | Friction Force | Half of Curve |

| Indenter: | Ft | | Ft | |
|---|---|---|---|---|
| Silicone Lens | [mN] | μ | [mN] | μ |
| Untreated Cartridge | 68.1 | 2.73 | 64.5 | 2.58 |
| Untreated Cartridge | 68.3 | 2.73 | 59.8 | 2.41 |
| Cartridge Treated With | 76.4 | 3.06 | 72.2 | 2.89 |
| Steam Only | | | | |
| Cartridge Treated With | 21.9 | 0.88 | 19.3 | 0.77 |
| CO₂ Only | | | | |
| Cartridge Treated With | 16.4 | 0.66 | 12.3 | 0.49 |
| Humidified CO₂ | | | | |
| Cartridge Treated With | 12.9 | 0.49 | 10.5 | 0.42 |
| Humidified CO₂ | | | | |
| Cartridge Treated With | 12.9 | 0.49 | 11.0 | 0.44 |
| Humidified CO₂ | | | | |
| Cartridge Treated With | 12.0 | 0.48 | 11.7 | 0.47 |
| Steam and CO₂ | | | | |
| Cartridge Treated With | 15.1 | 0.60 | 10.8 | 0.43 |
| Steam and CO₂ | | | | |
| Cartridge Treated With | 11.4 | 0.46 | 10.0 | 0.40 |
| Steam and CO₂ | | | | |
| Indenter: | — | — | — | — |
| Conical 10μ WC | | | | |
| Cartridge Treated With | — | — | 1.00 | 0.20 |
| Steam Only | | | | |
| Cartridge Treated With | — | — | 0.80 | 0.16 |
| CO₂ Only | | | | |
| Cartridge Treated With | — | — | 0.0 | 0.0 |
| Humidified CO₂ | | | | |
| Cartridge Treated With | — | — | 0.0 | 0.0 |
| Humidified CO₂ | | | | |

Ft = friction force
mN = milli-newton
μ = coefficient of friction

The anti-scratch slippery test shows how the lubricity or slickness of the lens cartridge is improved after treatment. Various types of treatments are listed in the table, and each shows an improvement over tests performed on cartridges before treatment in accordance with the methods of the present invention. Using an indenter with a silicone intraocular lens mounted on the diamond tip of the indenter to simulate the frictional force between a silicone intraocular lens and the cartridge in actual use, Ft for untreated cartridges was observed to be approximately 68 milli-newtons. Treating a cartridge with steam only resulted in a measurement of about 76 milli-newtons; this indicates that a cartridge treated with steam only was less slippery than an untreated cartridge, and would require more force to eject an intraocular lens from such a cartridge. In contrast, cartridges treated using a variety of methods such as steam and carbon dioxide gas and humidified carbon dioxide yield surfaces with substantial increase in slipperiness, as determined by the substantially reduced frictional force measured while the lens covered indenter was drawn across the surfaces of the treated cartridges. For example, treating a cartridge with humidified carbon dioxide gave an Ft of about 12.9 to 16, with similar results being achieved for surfaces treated with steam and carbon dioxide gas.

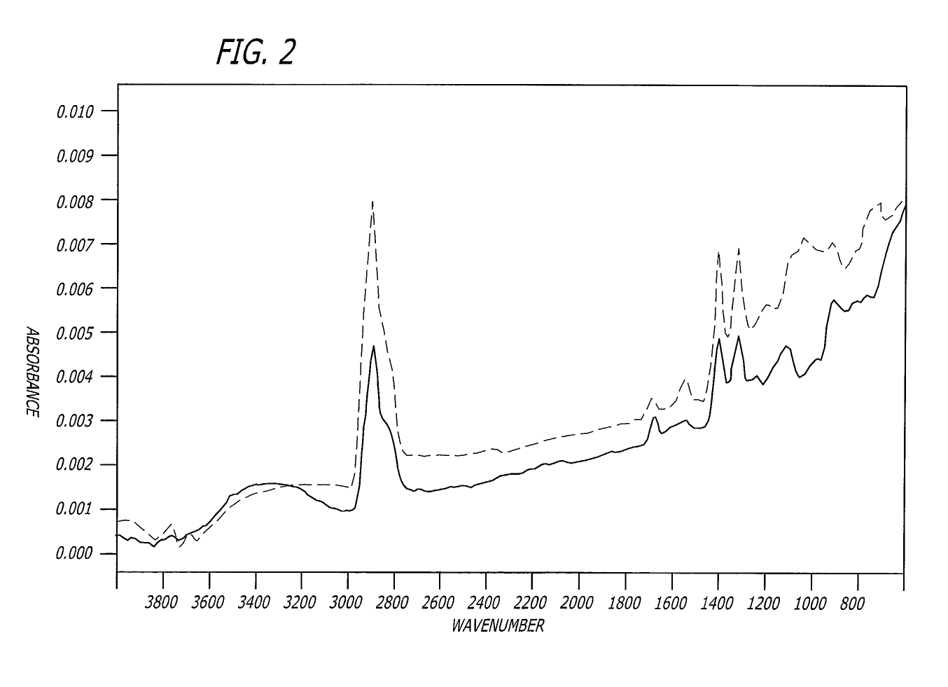

Tests were also conducted to prove that GMS molecules migrated to the surface regions of the polypropylene lens cartridges and that trace amounts of stearyl-carbonic acid formed during the treatment process. For this test, a FT-IR spectrum of a polypropylene lens cartridge before treatment (st45s wing ctrl) was prepared under nitrogen condition, followed by preparing another FT-IR spectrum of the same polypropylene lens cartridge (st45s wing post CO2 test (2)) after performing the second treatment procedure as described above. The FT-IR spectrum is shown in FIG. 2. By comparing the two FT-IR spectrums, it can be seen that peaks of OH or NH shift from 3400 to 3268 nm, indicating that more hydrogen bonds formed after the treatment. Peaks of methylene groups of GMS and stearylamide increased significantly (see 2917 nm and 1372 nm), indicating that there are more GMS and stearylamide molecules at the surface regions of the lens cartridge after treatment. Peaks of some carbonyl groups have shifted from 1728 to 1595 mn, also indicating that more hydrogen bonds have been formed. There is a peak of bending absorption of carbonic acid at 1256 nm indicating that trace amounts of carbonic acid have formed after treatment, and a peak of bending absorption of stearylamide at 808 nm indicating that GMS and stearylamide or distearylamide molecules have migrated to the surface regions of the polypropylene lens cartridge.

Test were also conducted to show that GMS molecules migrated to the surface region of the polypropylene cartridges and, once at the surface, formed micelles which resulted in increased hydrophilicity of the surfaces. FIGS. 3A and 3B are photographs showing the contact angle of a droplet of water with the surface. Such testing is well known by those skilled in the art to provide a measure of the relative hydrophilicity or hydrophobicity of a surface. As shown in FIGS. 3A and 3B, the contact angle observed in FIG. 3A is less, meaning that the meniscus between the droplet and the surface is more curved, indicating greater hydrophilicity that the untreated surface shown in FIG. 3B.

These methods can be used to restore or increase the surface lubricity of a medical device used to deliver implants into a human or animal body. In particular, lens cartridges with increased surface lubricity can be used to deliver intraocular lenses or intraocular contact lenses into the human eye for cataract surgery or myopia/hyperopia correction.

While several specific embodiments of the invention have been illustrated and described, it will be apparent that various modifications can be made without departing from the spirit and scope of the invention. Accordingly, it is not intended that the invention be limited, except as by the appended claims.

We claim:

1. A method for increasing the lubricity of a lens cartridge, comprising:
placing a lens cartridge into a chamber;
injecting a treatment gas into the chamber for a selected period of time; and
removing the lens cartridge from the chamber.

2. The method of claim 1, wherein removing the lens cartridge from the chamber further comprises placing the lens cartridge in a hermetically sealable package and sealing the package.

3. The method of claim 1, wherein the treatment gas includes a mixture of steam and carbon dioxide gas.

4. The method of claim 1, wherein the treatment gas is humidified carbon dioxide.

5. The method of claim 1, wherein the treatment gas includes a mixture of steam and another gas selected from the group of carbon dioxide, sulfur dioxide and nitrogen dioxide.

6. The method of claim 5, wherein the another gas is a combination of two or more gases selected from the group of carbon dioxide, sulfur dioxide and nitrogen dioxide.

7. The method of claim 1, wherein placing a lens cartridge into the chamber includes placing the lens cartridge in an unsealed hermetically sealable package and placing the unsealed hermetically sealable package containing the lens cartridge into the chamber.

8. The method of claim 7, wherein removing the lens cartridge from the chamber further comprises removing the hermetically sealable package containing the lens cartridge from the chamber and sealing the hermetically sealable package after the package is removed from the chamber.

9. The method of claim 1, wherein the chamber has an initial pressure within the chamber after the cartridge is placed in the chamber and wherein injecting a treatment gas includes
reducing the pressure within the chamber to a selected reduced pressure less than the initial pressure,
injecting the treatment gas until the pressure within the chamber is a selected pressure greater than the initial pressure.

10. The method of claim 1, wherein the chamber has an initial pressure within the chamber after the cartridge is placed in the chamber and wherein injecting a treatment gas includes
injecting the treatment gas until the pressure within the chamber is a selected pressure greater than the initial pressure, and
maintaining the pressure within the chamber at approximately the selected pressure during the selected period of time.

11. The method of claim 1, wherein the chamber has an initial pressure within the chamber after the cartridge is placed in the chamber and wherein injecting a treatment gas includes
maintaining the pressure within the chamber at approximately the initial pressure while injecting the treatment gas.

12. The method of claim 1, further comprising:
maintaining the chamber at a selected temperature while injecting the treatment gas.

13. The method of claim 12, wherein the temperature is in the range of approximately 30° C. to 60° C.

14. The method of claim 1, wherein injecting a treatment gas includes
injecting a mixture of steam and carbon dioxide gas for a first selected period of time,
halting the steam injection, and
continuing injecting steam for a second selected period of time.

15. The method of claim 9, wherein injecting the treatment gas includes
injecting a mixture of steam and carbon dioxide gas for a first selected period of time,
halting the steam injection, and
continuing injecting steam for a second selected period of time.

16. The method of claim 10, wherein injecting the treatment gas includes
injecting a mixture of steam and carbon dioxide gas for a first selected period of time,
halting the steam injection, and
continuing injecting steam for a second selected period of time.

17. The method of claim 11, wherein injecting the treatment gas includes
injecting a mixture of steam and carbon dioxide gas for a first selected period of time,
halting the steam injection, and
continuing injecting steam for a second selected period of time.

18. The method of claim 9, wherein the selected reduced pressure is in the range of 350 to 450 torr.

19. A method for increasing the lubricity of a lens cartridge, comprising:
placing a lens cartridge into an unsealed hermetically sealable package;
placing the unsealed hermetically sealable package containing the lens cartridge into an interior a chamber;
injecting a treatment gas into the chamber for a selected period of time;
maintaining the interior of the chamber at a selected temperature while the lens cartridge is within the chamber;
removing the unsealed hermetically sealable package containing the lens cartridge from the chamber; and
sealing the hermetically sealable package.

20. The method of claim 19, wherein injecting a treatment gas includes injecting a mixture of steam and carbon dioxide.

21. The method of claim 19, wherein injecting a treatment gas includes injecting humidified carbon dioxide.

22. The method of claim 19, wherein the selected temperature is in the range of approximately 20° C. to 60° C.

23. A method for increasing the lubricity of a lens cartridge, comprising:
placing a lens cartridge into an interior of a chamber, the chamber having an initial pressure after the lens cartridge is placed in the chamber;
maintaining the interior of the chamber at a selected temperature while the lens cartridge is within the chamber;
reducing the pressure within the chamber to a selected reduced pressure that is less than the initial pressure;
injecting a mixture of steam and carbon dioxide into the chamber until the pressure within the chamber is a selected pressure that is greater than the initial pressure;
maintaining the pressure in the chamber at the selected pressure for a first selected period of time;
halting injecting steam;
continuing injecting for a second selected period of time while maintain the chamber at the selected pressure; and
removing the lens cartridge from the chamber.

24. A method for increasing the lubricity of a lens cartridge, comprising:
placing a lens cartridge into an interior of a chamber, the chamber having an initial pressure after the lens cartridge is placed in the chamber;
maintaining the interior of the chamber at a selected temperature while the lens cartridge is within the chamber;
injecting a mixture of steam and carbon dioxide into the chamber until the pressure within the chamber is a selected pressure that is greater than the initial pressure;
maintaining the pressure in the chamber at the selected pressure for a first selected period of time;
halting injecting steam;
continuing injecting for a second selected period of time while maintain the chamber at the selected pressure; and
removing the lens cartridge from the chamber.

25. A method for increasing the lubricity of a lens cartridge, comprising:
placing a lens cartridge into an interior of a chamber, the chamber having an initial pressure after the lens cartridge is placed in the chamber;
maintaining the interior of the chamber at a selected temperature while the lens cartridge is within the chamber;
injecting a mixture of steam and carbon dioxide into the chamber while maintaining the pressure in the chamber at the initial pressure for a first selected period of time;
halting injecting steam;
continuing injecting for a second selected period of time while maintain the chamber at the initial pressure; and
removing the lens cartridge from the chamber.

26. A method for increasing the lubricity of a lens cartridge, comprising:
placing a lens cartridge into an interior of a chamber, the chamber having an initial pressure after the lens cartridge is placed in the chamber;
maintaining the interior of the chamber at a selected temperature while the lens cartridge is within the chamber;
injecting a treatment gas into the chamber until the pressure within the chamber is a selected pressure that is greater than the initial pressure;
maintaining the pressure in the chamber at the selected pressure for a selected period of time;
removing the lens cartridge from the chamber.

27. The method of claim 26, wherein the treatment gas is selected from the group consisting of carbon dioxide, sulfur dioxide and nitrogen dioxide.

28. The method of claim 26, wherein the treatment gas is a mixture of two or more gases selected from the group consisting of carbon dioxide, sulfur dioxide and nitrogen dioxide.

29. A method for increasing the lubricity of a lens cartridge, comprising:
placing a lens cartridge into an interior of a chamber, the chamber having an initial pressure after the lens cartridge is placed in the chamber;
maintaining the interior of the chamber at a selected temperature while the lens cartridge is within the chamber;
reducing the pressure within the chamber to a selected reduced pressure that is less than the initial pressure;
injecting a treatment gas into the chamber until the pressure within the chamber is a selected pressure that is greater than the initial pressure;
maintaining the pressure in the chamber at the selected pressure for a selected period of time;
removing the lens cartridge from the chamber.

30. The method of claim 29, wherein the treatment gas is selected from the group consisting of carbon dioxide, sulfur dioxide and nitrogen dioxide.

31. The method of claim 29, wherein the treatment gas is a mixture of two or more gases selected from the group consisting of carbon dioxide, sulfur dioxide and nitrogen dioxide.

32. A method for increasing the lubricity of a lens cartridge, comprising:
placing a lens cartridge into an interior of a chamber, the chamber having an initial pressure after the lens cartridge is placed in the chamber;
maintaining the interior of the chamber at a selected temperature while the lens cartridge is within the chamber;
reducing the pressure within the chamber to a selected reduced pressure that is less than the initial pressure;
injecting a treatment gas into the chamber until the pressure within the chamber is a selected pressure that is greater than the selected reduced pressure;
maintaining the pressure in the chamber at the selected pressure for a selected period of time;
removing the lens cartridge from the chamber.

33. The method of claim 32, wherein the treatment gas is selected from the group consisting of carbon dioxide, sulfur dioxide and nitrogen dioxide.

34. The method of claim 32, wherein the treatment gas is a mixture of two or more gases selected from the group consisting of carbon dioxide, sulfur dioxide and nitrogen dioxide.

35. A method for increasing the lubricity of a lens cartridge, comprising:
placing a lens cartridge into an interior of a chamber, the chamber having an initial pressure after the lens cartridge is placed in the chamber;
maintaining the interior of the chamber at a selected temperature while the lens cartridge is within the chamber;
injecting a treatment gas into the chamber while maintaining the pressure within the chamber at the initial pressure for a selected period of time;
removing the lens cartridge from the chamber.

36. The method of claim 35, wherein the treatment gas is selected from the group consisting of carbon dioxide, sulfur dioxide and nitrogen dioxide.

37. The method of claim 35, wherein the treatment gas is a mixture of two or more gases selected from the group consisting of carbon dioxide, sulfur dioxide and nitrogen dioxide.

38. A lens cartridge for use in a surgical lens inserting device for implantation of a deformable intraocular lens, comprising:
a lens holding portion;
a nozzle portion connected to and extending from the lens holding portion, the lens holding portion and the nozzle portion having a passageway extending therethrough, the passageway having an inner surface having an increased lubricity for assisting in moving the deformable intraocular lens through the passageway.

39. The lens cartridge of claim 38, wherein the lens holding portion and the nozzle portion are formed from glycerin monostearate modified polypropylene.

40. A method for increasing the surface lubricity of a lens cartridge, comprising:
providing a lens cartridge having an inner surface; and
injecting humidified carbon dioxide over the inner surface of the lens cartridge.

41. A cartridge for implanting a deformable intraocular lens into an eye of a patient, comprising:
a body portion formed from glycerin monostearate modified polypropylene, the body portion having a lens holding portion and a nozzle portion; and
a passageway having an inner surface extending through the body portion, the passage way having an opening disposed at an end of the nozzle portion for allowing ejection of a deformable intraocular lens from the passageway, the inner surface having increased lubricity formed by exposing the inner surface of the passage way to a treatment gas prior to mounting the deformable intraocular lens into the passageway.

42. A cartridge for implanting a deformable intraocular lens into an eye of a patient, comprising:
a body portion formed from polypropylene containing a second constituent, the body portion having a lens holding portion and a nozzle portion; and
a passageway having an inner surface extending through the body portion, the passage way having an opening disposed at an end of the nozzle portion for allowing ejection of a deformable intraocular lens from the passageway, the inner surface having increased lubricity formed by exposing the inner surface of the passageway to a treatment gas prior to mounting the deformable intraocular lens into the passageway such that the second constituent interacts with the treatment gas to increase the lubricity of the passageway.

43. A cartridge for implanting a deformable intraocular lens into an eye of a patient, comprising:
a body portion formed from polypropylene and a second constituent, the body portion having a lens holding portion and a nozzle portion; and
a passageway having an inner surface extending through the body portion, the passage way having an opening disposed at an end of the nozzle portion for allowing ejection of a deformable intraocular lens from the passageway, the inner surface having increased lubricity formed by exposing the inner surface of the passage way to humidified carbon dioxide prior to mounting the deformable intraocular lens into the passageway, the humidified carbon dioxide and the second constituent interacting to provide increased lubricity to the inner surface of the passageway.
